(19) United States
(12) Patent Application Publication
(10) Pub. No.: US 2011/0105976 A1
(43) Pub. Date: May 5, 2011

(54) HAND SANITIZING PATCH

(76) Inventor: Judd Berlin, Minneapolis, MN (US)

(21) Appl. No.: 12/921,253

(22) PCT Filed: Mar. 5, 2009
(86) PCT No.: PCT/US09/01407

§ 371 Date: Dec. 20, 2010

Related U.S. Application Data
(60) Provisional application No. 61/034,862, filed on Mar. 7, 2008.
(60) Provisional application No. 61/038,958, filed on Mar. 24, 2008.

(51) Int. Cl. A61F 13/02; A61M 35/00

(52) U.S. Cl. 602/48

(57) ABSTRACT

The invention provide a topical adhesive patch having a flexible backing having a front side and a back side; a first formulation positioned on at least a portion on the front side of the backing, in at least a portion on the front side of the backing, or on and in at least a portion on the front side of the backing; a second formulation positioned on at least a portion on the back side of the backing, in at least a portion on the back side of the backing, or on and in at least a portion on the back side of the backing; the first formulation comprises an adhesive; and the second formulation comprising an antibiotic agent, anti-fungal agent, anti-viral agent, or combination thereof, and uses therefor.

### Related Applications

This application claims the benefit of the filing date of U.S. Application Ser. No. 61/034,862, filed on Mar. 7, 2008, and U.S. Application Ser. No. 61/038,958, filed on Mar. 24, 2008, the disclosures of which are incorporated by reference herein.

### SUMMARY OF THE INVENTION

The invention provides a topical adhesive patch. The patch includes a flexible backing having a front side and a back side; a first formulation positioned on at least a portion on the front side of the backing, in at least a portion on the front side of the backing, or on and in at least a portion on the front side of the backing; a second formulation positioned on at least a portion on the back side of the backing, in at least a portion on the back side of the backing, or on and in at least a portion on the back side of the backing. The first formulation includes an adhesive and the second formulation includes an antibiotic agent, anti-fungal agent, anti-viral agent, or any combination thereof.

Also provided is a method of preventing or inhibiting a pathogen related disease, e.g., a bacterial disease, a fungal disease, a viral disease, or any combination thereof, in a mammal at risk of such disease. The method includes contacting a skin surface of the mammal with a topical adhesive patch of the invention having an amount of one or more anti-pathogen agents effective to prevent or inhibit the disease. In one embodiment, the agent prevents or inhibits infection by the pathogen. In one embodiment, the agent prevent or inhibits replication of the pathogen. In one embodiment, the amount is a bacteriocidal amount. In one embodiment, the amount is a bacteriostatic amount.

### BRIEF DESCRIPTION OF THE DRAWINGS

FIG. 1 illustrates the front side of an adhesive patch of the present invention with a release liner attached to the patch.

FIG. 2 illustrates the back side of an adhesive patch of the present invention with a release liner attached to the patch, where the patch is partially detached from the release liner.

FIG. 3 illustrates one embodiment in which the adhesive patch of the present invention has an oval or elliptical shape.

FIG. 4 illustrates one embodiment in which the adhesive patch of the present invention covers a portion of the dorsal side of a hand.

FIG. 5 illustrates one embodiment in which the adhesive patch of the present invention covers the entire dorsal side of a hand.

FIG. 6 illustrates one embodiment in which the adhesive patch of the present invention covers the dorsal side of a hand from the wrist up to the fingernails (not including fingernails).

FIG. 7 illustrates an enlarged cross-sectional view of specific patch of the present invention.

FIG. 8 illustrates the diffusion of various components within an enlarged cross-sectional view of specific patch of the present invention.

### DETAILED DESCRIPTION OF THE INVENTION

The present invention provides a unique adhesive vehicle. The vehicle has pressure sensitive adhesive qualities due to its composition and viscoelastic nature. The adhesive formulation is hydrophilic and therefore, water may dissolve into or evaporate from the adhesive, depending on the conditions to which it is exposed. This water exchange capability implies that if the adhesive formulation is on, e.g., a suitably porous backing and is applied to the skin, it will not be occlusive as most drug delivery patches are. The occlusive nature of conventional drug delivery patches is thought to play an important role in enhancing drug accessibility, but also often results in greater incidence of skin irritation. The relatively low occlusiveness of a specific adhesive patch in a specific embodiment of the present invention may be envisioned to be a special adhesive ointment or gel which is water-breathable, such as a water washable or water soluble ointment or gel.

The present invention provides an ointment or gel on a backing. The ointment or gel may include an effective, known, and safe amount of an antiviral agent, an antibiotic agent, an antifungal agent, and other medicaments useful for preventing communicable infections diseases. The backing is pliable and/or stretchable. Since the backing may be porous and/or vapor permeable, many consumers typically refer to the device as a “patch,” a Askin patch,@ or an “adhesive skin patch.” As such, the device (i.e., the ointment or gel on the backing) will herein be referred to as a patch, a skin patch, and/or an adhesive skin patch. It is appreciated that those skilled in the art understand that the term “patch” is used to refer to the device and is not otherwise limiting in any manner.

As used herein, “holdout” refers to the physical properties of a backing, relating to the ability of a specific class of gels or ointments to penetrate, cross-link, wet, and/or disinfect within the matrix of the backing. A specific class of gels or ointments may or may not be able to penetrate a given backing. Upon penetration of a gel or ointment into a backing, the gel or ointment will cross-link, wet, or disinfect in the backing. As such, the holdout properties are the ability of the gel or ointment to affect the degree of penetration, cross-linking, wetting, and/or disinfecting within the matrix of the backing. Those backings with superior holdout properties may prevent, decrease, or lessen the likelihood of the ointment or gel from wetting the backing; may increase the likelihood of the ointment or gel to cross-link within the matrix of the backing; may increase the likelihood of the ointment or gel to disinfect within the matrix of the backing; and/or may prevent, decrease, or increase the likelihood of the ointment or gel to partially penetrate the matrix of the backing.

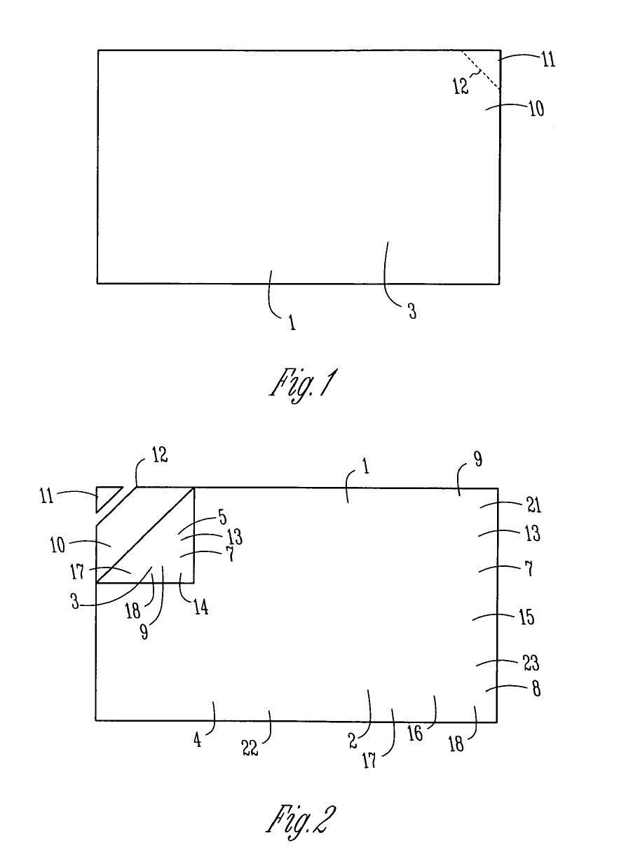

Referring to FIGS. 1-8, an exemplary adhesive patch 1 of the present invention is provided.

Backing

The backing 2 is defined by a front side 3 (the side exposed to the skin during use) and a back side 4 (the side exposed to the environment during use). The backing 2 should be nonirritating to human skin. The backing 2 is a self-supporting sheet of water soluble or water insoluble, polymeric or natural material that provides strength and integrity for the first formulation 5 and the second formulation 24. The backing 2 of the adhesive patch 1 may be vapor permeable. The backing 2 may also be porous, since porosity provides openings for receiving the first formulation 5 and second formulation 24, and it helps to assure that the adhesive skin patch 1 is vapor permeable. Specifically, the backing 2 may retain the formulation 5 while allowing moisture from the skin to pass. Alternatively, the backing 2 may be non-porous. The backing 2 may have any suitable thickness, provided the suitable thickness allows for a flexible, bendable, pliable, vapor permeable, and/or a stretchable sheet of water insoluble porous material. Specifically, the thickness of the backing 2 may be about 0.001 mm to about 5.0 mm, about 0.001 mm to about 3.0 mm, or about 0.025 mm to about 1.25 mm.

The backing 2 may be manufactured from any suitable material, provided the suitable material forms a flexible, bendable, pliable, and/or stretchable backing 2. The backing 2 includes a flexible porous or non-porous sheet of water soluble or water insoluble material that provides support for the adhesive skin patch 1. The backing 2 may include water soluble or water insoluble polymeric fibers, a porous film, or any other kind of matrix with spaces within the matrix. A specific backing 2 is a lightweight, porous, pliable strip composed of a nonwoven fabric of polymeric or natural fibers such as polyester, cotton or cellulose fibers bonded together with a sizing resin. The backing 2 may be woven or nonwoven. In one embodiment, the backing 2 includes nonwoven fabric. Specifically, the backing 2 may include polyester fibers, polyurethane fibers, polyolefin fibers, polyamide fibers, natural fibers, cotton fibers, copolyester, copolyester fibers, cellulose acetate fibers, polycellulose fibers, or any mixture thereof. Additional stable, water insoluble flexible sheet materials and methods for manufacturing the suitable backings 2 are disclosed, e.g., in U.S. Pat. No. 4,675,009; U.S. Pat. No. 5,536,263; U.S. Pat. No. 4,696,854; U.S. Pat. No. 5,741,510, and references cited therein, and are suitable as backings 2 according to the present invention. The infusion of the formulation 5 and second formulation 24 into the backing 2 may be accomplished, e.g., with the use of a continuous process mixer, as disclosed, e.g., in U.S. Pat. No. 5,536,263, and references cited therein; or as discussed herein.

Alternatively, the backing 2 may be a non-woven backing 2 that is treated by coating: the front side 3 of the backing 2, the back side 4 of the backing 2, or both the front side 3 and back side 4 of the backing 2; with a silicone-containing compound, a fluorocarbon solution, or a combination thereof. Suitable silicone-containing compounds include, e.g., polydimethyl siloxanes, dialkylsiloxanes, dimethylsiloxo vinyl alkenes, dialkylsiloxo vinyl alkenes, dimethylsiloxo acrylates, dialkylsiloxo acrylates, vinyl terminated polydimethylsiloxane, and vinyl terminated polydialkylsiloxane. The exemplary silicone-containing compounds are commercially available from, e.g., Goldschmidt Chemical Corp. (Essen, Germany); GE Silicones (Waterford, N.Y.); Wacker Silicone Corp. (Adrian, Mich.); and Dow Corning Corp. (Midland, Mich.).

The backing 2 may be manufactured from a suitable non-woven fabric that is commercially available from, e.g., Freudenberg Faservliesstoffe KG (Weinham, Germany); Sontara Technologies (division of DuPont Corporation) (Old Hickory, Tenn.); Lystil S.A. (Brignoud Cedex, France); Dexter Nonwovens (Windsor Locks, Conn.); and Chicopee (New Brusnwick, N.J.). Other commercial vendors that supply suitable non-woven fabrics may be found at the Technical Textile website (http://www.technical-textiles.net/technical-textiles-index/orgL.htm).

The use of a treated backing 2, such as a fluorocarbon treated non-woven backing, typically increases the yield of an adhesive skin patch 1 of the present invention. The use of a backing material that has been treated with a sizing agent may allow for the effective control of the rate of penetration, such that a cosmetically acceptable gel 25 or cosmetically acceptable ointment 26 has solidified after it has begun to penetrate the back side 4 of the backing 2, but before it has passed completely through the back side 4 of the backing 2. In addition, the use of a backing material that has been treated with a sizing agent may allow for the effective control of the distance to which the cosmetically acceptable gel 25 or cosmetically acceptable ointment 26 easily penetrates before solidifying. Increasing the control of the rate at which the cosmetically acceptable gel 25 or cosmetically acceptable ointment 26 penetrates the backing 2 typically improves the overall yield of the production process by reducing the amount of material which may be discarded because the front side 3 of the backing 2 has become too tacky for either processing or for consumer acceptance.

At least a portion of the backing 2 may be treated with a sizing agent 20 such that the portion of the backing 2 that is treated with the sizing agent 20 has a surface energy of about 20 dynes/cm² to about 65 dynes/cm². Specifically, the portion of the backing 2 that is treated with the sizing agent 20 may have a surface energy of about 27 dynes/cm² to about 56 dynes/cm². The sizing agent 20 may lower the surface energy of the portion of the backing 2 that is treated with the sizing agent 20. Any suitable sizing agent 20 may be employed, provided the portion of the backing 2 that is treated with the sizing agent 20 has a surface energy of about 20 dynes/cm² to about 65 dynes/cm². Suitable sizing agents 20 include, but are not limited to, e.g., fluorocarbon solutions, silicone-containing compounds, and combinations thereof. Specifically, the backing 2 may be a non-woven backing 2 that is treated with a fluorocarbon. For example, the fluorocarbon treated backing 2 may be, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, or Vilmed M1578 FH; which are all commercially available from Freudenberg Faservliesstoffe KG (Weinham, Germany). Alternatively, the silicone treated backing 2 may be a non-woven backing 2 that is coated with one or more silicone-containing compounds, e.g., a polydimethyl siloxane, a dialkylsiloxane, a dimethylsiloxo vinyl alkene, a dialkylsiloxo vinyl alkenes, a dimethylsiloxo acrylate, a dialkylsiloxo acrylate, a vinyl terminated polydimethylsiloxane, and a vinyl terminated polydialkylsiloxane.

At least a portion of the backing 2 may be treated with the sizing agent 20. The portion of the backing 2 that is treated with the sizing agent 20 may be that portion of the backing 2 that may typically include the first formulation 5 and the second forumulation 24. The entire surface of the back side 4 of the backing 2 may be treated with the sizing agent 20 or a portion of the surface of the back side 4 of the backing 2 may be treated with the sizing agent 20. In one embodiment, the entire surface of the back side 4 of the backing 2 may be treated with the sizing agent 20. In addition to the surface of the back side 4 of the backing 2 being treated with the sizing agent 20, the sizing agent 20 may penetrate at least a portion of the underlying surface (e.g., one-tenth to about nine-tenths the thickness, or about one-fourth to about nine-tenths the thickness) of the backing 2. Specifically, the sizing agent 20 may penetrate the entire underlying surface of the backing 2.

Suitable fluorocarbon solutions include, but are not limited to, e.g., Vilmed M1585 W/HYJ, Vilmed M1585H/HYJ, Vilmed M1586 W/HYJ, Vilmed M1586 H/HYj, Vilmed M1570J, Vilmed M1573 FJ, Vilmed M1573 FHJ, Vilmed M1577 FJ, Vilmed M1578FJ, and Vilmed M1578 FHJ; which are all commercially available from Freudenberg Faservliesstoffe KG (Weinham,

Germany).

Alternatively, the fibers of the backing 2 may be interlocked mechanically by air or water.

As shown in FIGS. 1-2 and 7-8, the backing 2 may include a front side 3 and a back side 4. The adhesive skin patch 1 may include a first formulation 5 located in at least a portion of the front side 3 of the backing 2, on at least a portion of the front side 3 of the backing 2, or on and in at least a portion of the front side 3 of the backing 2. As such, the formulation 5 may be located on the entire surface of the front side 3 of the backing 2 or the formulation 5 may be located on a portion of the surface of the front side 3 of the backing 2.

In one embodiment, the first formulation 5 may be located on the entire surface of the front side 3 of the backing 2. In addition to being located on the surface of the front side 3 of the backing 2, the first formulation 5 may be located in at least a portion of the underlying surface of the front side 3 of the backing 2 (e.g., the first formulation 5 may be partially embedded into the backing 2).

The first formulation 5 may penetrate a substantial portion of the front side 3 of the backing 2, as disclosed, e.g., in U.S. Pat. No. 5,536,263, and references cited therein. For example, the first formulation 5 may penetrate about one-tenth to about nine-tenths the thickness of the backing 2, or about one-fourth to about nine-tenths the thickness of the backing 2. As such, the first formulation 5 may be partially embedded into the backing 2. In one embodiment, the first formulation 5 may be located on the entire front side 3 of the backing 2 and partially in the front side 3 of the backing 2 (e.g., the first formulation 5 is partially embedded into the backing 2).

Alternatively, a portion of the front side 3 of the backing 2 may include the first formulation 5 and other portions of the front side 3 of the backing 2 may include any suitable and effective combination of the pressure sensitive adhesive 14 and, optionally, the solvent 13. For example, a central circular portion of the front side 3 of the backing 2 may include the first formulation 5 while the remaining portions of the front side 3 of the backing 2 include only the pressure sensitive adhesive 14. The first formulation 5, when partially embedded into the front side 3 of the backing 2, may impart strength and structure into the adhesive patch 1. For example, when the first formulation 5 is partially embedded into the backing 2, the likelihood that the adhesive patch 1 tears apart when separated from the release liner 10 or when removed from the skin after use, is lowered.

The adhesive skin patch 1 includes a second formulation 24 located in at least a portion of the back side 4 of the backing 2, on at least a portion of the back side 4 of the backing 2, or on and in at least a portion of the back side 4 of the backing 2. As such, the second formulation 24 may be located on the entire surface of the back side 4 of the backing 2 or the second formulation 24 may be located on a portion of the surface of the back side 4 of the backing 2.

In one embodiment, the second formulation 24 may be located on the entire surface of the back side 4 of the backing 2. In addition to being located on the surface of the back side 4 of the backing 2, the second formulation 24 may be located in at least a portion of the underlying surface of the back side 4 of the backing 2 (e.g., the second formulation 24 may be partially embedded into the backing 2).

The second formulation 24 may penetrate a substantial portion of the front back 4 of the backing 2, as disclosed, e.g., in U.S. Pat. No. 5,536,263, and references cited therein. For example, the second formulation 24 may penetrate about one-tenth to about nine-tenths the thickness of the backing 2, or about one-fourth to about nine-tenths the thickness of the backing 2. As such, the second formulation 24 may be partially embedded into the backing 2. In one embodiment, the second formulation 24 may be located on the entire back side 4 of the backing 2 and partially in the back side 4 of the backing 2 (e.g., the second formulation 24 is partially embedded into the backing 2).

Alternatively, a portion of the back side 4 of the backing 2 may include the second formulation 24 and other portions of the back side 24 of the backing 2 may include any suitable and effective solvent 13. For example, a central circular portion of the back side 4 of the backing 2 may include the second formulation 24 while the remaining portions of the back side 4 of the backing 2 include only the solvent 13. The second formulation 24, when partially embedded into the back side 4 of the backing 2, may impart strength and structure into the adhesive patch 1. For example, when the second formulation 24 is partially embedded into the backing 2, the likelihood that the adhesive patch 1 tears apart when separated from the release liner 10 or when removed from the skin after use, is lowered.

When the adhesive skin patch 1 is placed upon the skin of a patient (e.g., human), the first formulation 5 may be in continuous contact with the skin surface of the patient. In one embodiment, the adhesive skin patch 1, upon contact with skin, may allow the skin to breathe. In one embodiment, the adhesive skin patch 1, upon prolonged contact with skin, holds in place the first formulation 5 and second formulation 24, and permits the skin to breathe over prolonged periods of time typically experienced with the use of the patch, e.g., up to about 7 days, up to about 24 hours, up to about 12 hours, up to about 8 hours, or up to about 6 hours.

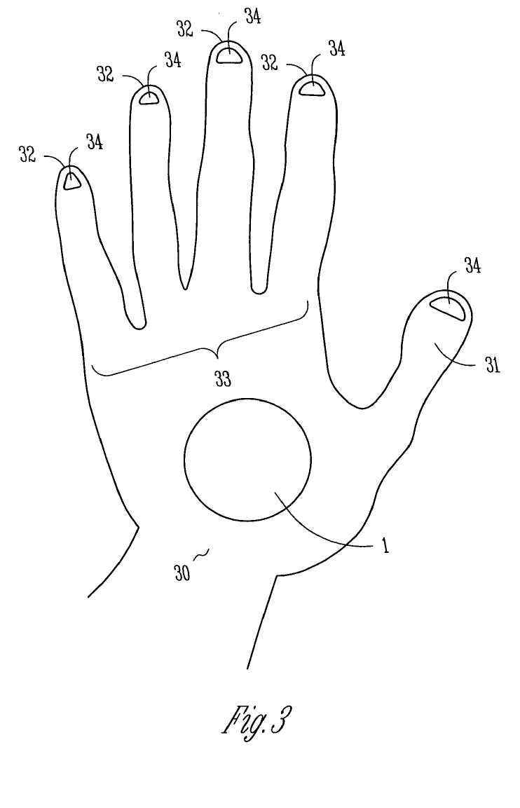

As shown in FIG. 1, the adhesive skin patch 1 may be reversibly attached to a release liner 10. The release liner 10 helps to maintain the adhesiveness of the adhesive skin patch 1 prior to use, such as during manufacturing, packaging, shipping, and/or storage. Any suitable release liner 10 may be employed for use in the present invention. Suitable release liners 10 are readily known to those of skill in the art. See, e.g., U.S. Pat. No. 4,675,009; U.S. Pat. No. 5,536,263; U.S. Pat. No. 4,696,854; U.S. Pat. No. 5,741,510, and references cited therein for further descriptions of release liners 10 useful in the present invention. The release liner 10 may include a perforation 12 that allows the tab section 11 of the release liner 10 to be removed (see, FIGS. 1-2). Removal of the tab section 11 of the release liner 10 may allow the adhesive skin patch 1 to be removed from the release liner 10 with relative ease.

In one embodiment, the patch 1, upon contact with skin, may allow the skin to breathe. In one embodiment, the patch 1, upon prolonged contact with skin, holds in place the first formulation 5 and second formulation 24, and permits the skin to breathe over prolonged periods of time typically experienced with the use of the patch, e.g., up to about 7 days, up to about 24 hours, up to about 12 hours, up to about 8 hours, or up to about 6 hours.

The backing 2 may be a porous or non-porous, self-supporting sheet of water insoluble or water soluble, polymeric or natural material that provides strength and integrity for the first formulation 5 and second formulation 24. For example, the backing 2 may be water insoluble polymeric fibers, open cell foam backing (e.g., polyurethane, polyvinyl chloride, or polyethylene), a porous film, or any other kind of matrix with spaces within the matrix. In one embodiment, the backing 2 may include polyester, polyurethane, polyolefin, polyamide fibers, natural fibers, cotton fibers, polycellulose fibers, or any mixture thereof.

A specific backing 2 is a lightweight, porous, pliable strip composed of a nonwoven fabric of polymeric or natural fibers such as polyester, cotton or cellulose fibers bonded together with a sizing resin. Additional stable, water insoluble flexible sheet materials are disclosed, e.g., in U.S. Pat. No. 4,675,009; U.S. Pat. No. 5,536,263; U.S. Pat. No. 4,696,854; U.S. Pat. No. 5,741,510, and references cited therein, and are suitable as backings according to the present invention. The infusion of the first formulation 5 and second formulation 24 into the backing 2 may be accomplished with the use of a continuous process mixer, as disclosed, e.g., in U.S. Pat. No. 5,536,263, and references cited therein.

As shown in FIGS. 1-2 and 7-8, the patch 1 may reversibly attached to a release liner 10. The release liner 10 helps to maintain the adhesiveness of the patch 1 prior to use, such as during manufacturing, packaging, shipping, and/or storage. Any suitable release liner 10 may be employed for use in the present invention. Suitable release liners 10 are readily known to those of skill in the art. See, e.g., U.S. Pat. No. 4,675,009; U.S. Pat. No. 5,536,263; U.S. Pat. No. 4,696,854; U.S. Pat. No. 5,741,510, and references cited therein for further descriptions of release liners 10 useful in the present invention. The release liner 10 may include a perforation 12 that allows the tab section 11 of the release liner 10 to be removed (see, FIGS. 1-2). Removal of the tab section 11 of the release liner 10 may allow the patch 1 to be removed from the release liner 10 with relative ease.

In one specific embodiment, the back side 4 of the backing 2 of the patch 1 may be relatively dry to the touch, such that upon contact, e.g., with a skin surface or article of clothing, no appreciable or significant amount of liquid, gel, ointment, fluid, lotion, and the like, present in the back side 4 of the backing 2 of the patch 1 is transferred there from and deposited upon the skin surface or article of clothing.

In another embodiment, the back side 4 of the backing 2 of the patch 1 may be relatively wet to the touch, such that upon contact, e.g., with a skin surface, an appreciable and significant amount of liquid, gel, ointment, fluid, lotion, and the like, present in the back side 4 of the backing 2 of the patch 1 is transferred there from and deposited upon the skin surface. In such an embodiment, the patient may use the adhesive patch 1 by rubbing his/her hands across the back side 4 of the backing 2 of the patch 1. Rubbing the back side 4 of the backing 2 of the patch 1 causes a portion of the second formulation 24 to be deposited on the hand that is rubbing. In such an embodiment, the second formulation 24 may include an appreciable and significant amount of liquid, gel, ointment, fluid, lotion, and the like, that upon rubbing, may be transferred there from and deposited upon the skin surface.

The second formulation 24 may then be deposited on the non-rubbing hand or another skin surface through subsequent rubbing. In one embodiment, the patient may wear two adhesive patches 1, one on the dorsal aspect of each hand.

First Formulation

As shown in FIGS. 1-2 and 7-8, the backing 2 may include a front side 3 and a back side 4. The patch 1 may include first formulation 5 located in at least a portion of the front side 3 of the backing 2, located on at least a portion of the front side 3 of the backing 2, or located on and in at least a portion of the front side 3 of the backing 2. In one embodiment, the first formulation 5 is located on the entire front side 3 of the backing 2 and partially in the front side 3 of the backing 2 (e.g., the first formulation 5 is partially embedded into the backing 2).

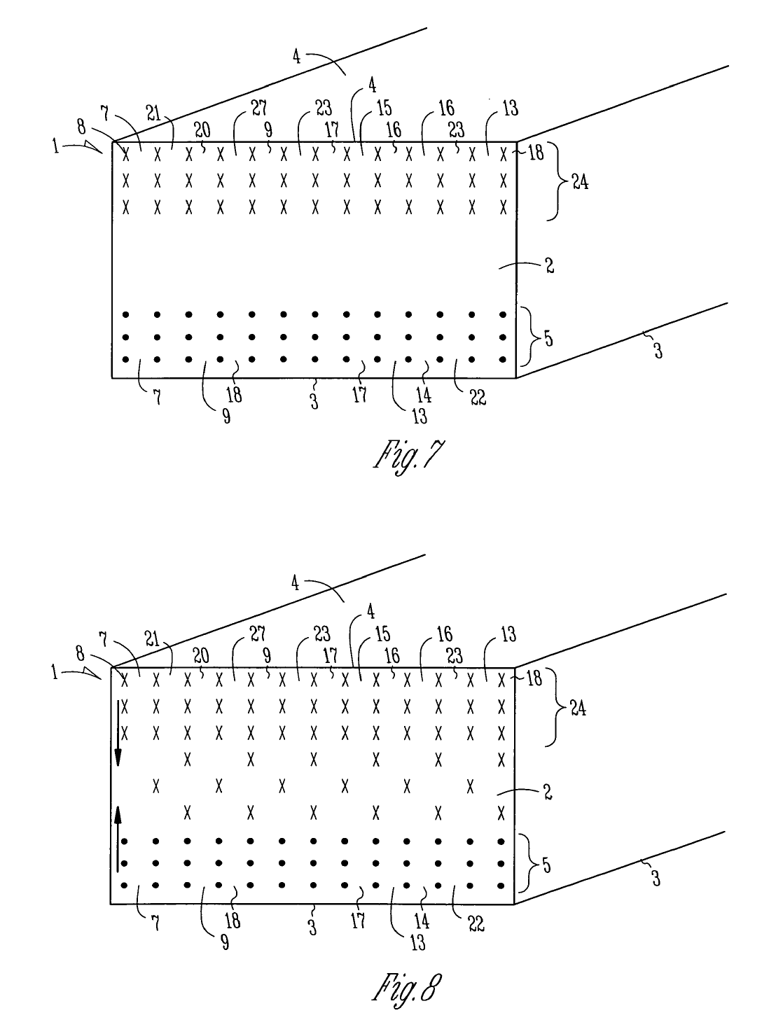

The first formulation 5 may be positioned on and in any portion of the front side 3 of the backing 2. The first formulation 5 may be positioned in a portion of the front side 3 of the backing 2 (e.g., the first formulation 5 penetrates a substantial portion of the front side 3 of the backing 2) as disclosed in, e.g., U.S. Pat. No. 5,536,263, and references cited therein. For example, the first formulation 5 may penetrate a substantial portion of the front side 3 of the backing 2, e.g., typically between about one-fourth to about nine-tenths the thickness of the backing 2. The penetration of the first formulation 5 into the backing 2 may be seen in FIGS. 7-8.

In one embodiment, the first formulation 5 may be positioned on the entire front side 3 of the backing 2. In this latter configuration, the first formulation 5 is in continuous contact with the entire front side 3 of the backing 2. When the adhesive skin patch 1 is placed upon the skin surface of a patient, the first formulation 5 is in continuous contact with the skin surface of the patient.

Alternatively, a portion of the front side 3 of the backing 2 may contain the first formulation 5 and other portions of the front side 3 of the backing 2 may contain any combination of the adhesive 14, and, optionally, the solvent 13. For example, a central circular portion of the front side 3 of the backing 2 may contain the first formulation 5 while the remaining portions of the front side 3 of the backing 2 contains only the adhesive 14.

The first formulation 5 may include an adhesive 14 and, optionally, one or more of the following components: a solvent 13, an antimicrobial agent 7, one or more polymers 9, a humectant 17, a topical moisturizer 18, and one or more polyhydric alcohols 22.

The first formulation 5 may remain stable over the period of time typically experienced with the manufacturing, packaging, shipping, and/or storage of the adhesive skin patch 1, e.g., up to about a month, up to about a year, or up to about two years.

Second Formulation

As shown in FIGS. 1-2 and 7-8, the backing 2 may include a front side 3 and a back side 4. The patch 1 may include second formulation 24 located in at least a portion of the back side 4 of the backing 2, located on at least a portion of the back side 4 of the backing 2, or located on and in at least a portion of the back side 4 of the backing 2. In one embodiment, the second formulation 24 is located on the entire back side 4 of the backing 2 and partially in the back side 4 of the backing 2 (e.g., the second formulation 24 is partially embedded into the backing 2).

The second formulation 24 may be positioned on and in any portion of the back side 4 of the backing 2. The second formulation 24 may be positioned in a portion of the back side 4 of the backing 2 (e.g., the second formulation 24 penetrates a substantial portion of the back side 4 of the backing 2) as disclosed in, e.g., U.S. Pat. No. 5,536,263, and references cited therein. For example, the second formulation 24 may penetrate a substantial portion of the back side 4 of the backing 2, e.g., typically between about one-fourth to about nine-tenths the thickness of the backing 2. The penetration of the second formulation 24 into the backing 2 may be seen in FIGS. 7 and 8.

In one embodiment, the second formulation 24 may be positioned on the entire back side 4 of the backing 2. In this latter configuration, the second formulation 24 may be in continuous contact with the entire back side 4 of the backing 2.

Alternatively, a portion of the back side 4 of the backing 2 may contain the second formulation 24. For example, a central circular portion of the back side 4 of the backing 2 may contain the second formulation 24.

The second formulation 24 may include, for example, a solvent 13, and an antiviral agent 15, an antibiotic agent 16, and/or an antifungal agent 23. The second formulation 24 may also include one of the following optional components: a topically suitable oil 27, an antimicrobial agent 7, a fragrance 8, one or more polymers 9, a topically suitable oil 27, a topical moisturizer 18, one or more essential oils 21, and a sizing agent 20.

The second formulation 24 may remain stable over the period of time typically experienced with the manufacturing, packaging, shipping, and/or storage of the adhesive skin patch 1, e.g., up to about a month, up to about a year, or up to about two years.

Antiviral Agent

As used herein, an Aantiviral agent@ is a compound or combination of compounds that weakens or abolishes the action of a virus. Stedman's Medical Dictionary, 25th Ed., illustrated, Williams & Wilkins, Baltimore, Md., p. 101 (1990). Any suitable antiviral agent 15 may be employed, provided the antiviral agent 15 effectively treats or inhibits a viral infection and/or replication and the antiviral agent 15 remains stable in the second formulation 24. In one embodiment, the stability is over a prolonged period of time, e.g., up to about 2 years, up to about 1 year, or up to about 6 months, typically experienced in the manufacturing, packaging, shipping, and/or storage of the patch 1.

Suitable antiviral agents are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; and references cited therein. Suitable antiviral agents 15 include, e.g., zinc, lysine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, viracea2, cytovene, famciclovir, valaciclovir, penciclovir, nonoxynol-9, pharmaceutically acceptable salts thereof, and combinations thereof. Additional suitable antiviral agents 15 include, e.g., a hypochloride, a hypochloride generating compound, a peroxide, a peroxide generating compound, an organic halide, an organic halide generating compound, or a combination thereof.

The antiviral agent 15 may be present in any appropriate and suitable amount, provided the amount of antiviral agent 15 is effective to treat or inhibit a viral infection and/or replication and the amount of antiviral agent 15 remains stable in the second formulation 24 over a prolonged period of time. Typically, the antiviral agent 15 may be present in about 0.01 wt. % to about 99.9 wt. % of the second formulation 24. The amount of antiviral agent 15 present in the second formulation 24 may depend upon the specific compound or compounds employed as the antiviral agent 15. For example, lysine hydrochloride may be present up to about 99.9 wt. % of the second formulation 24, up to about 50 wt. % of the second formulation 24, or up to 20 wt. % of the second formulation 24. In one embodiment, the amount of antiviral agent 15 employed in the second formulation 24 complies with FDA regulations.

The antiviral agent 15 may be located on and in any portion of the second formulation 24, which is located on the back side 4 of the backing 2. In one embodiment, the antiviral agent 15 may be located on and in the entire portion of the second formulation 24. When the adhesive skin patch 1 is placed upon the skin of a patient (e.g., human), the antiviral agent 15 may be in continuous contact with the skin surface of the patient.

Antibiotic Agent

The second formulation 24 may include one or more suitable antibiotic agents. As used herein, an “antibiotic agent” is any compound having activity against either Gram-positive or Gram-negative organisms (i.e., inhibits the growth or destroys the development of either Gram-positive or Gram-negative organisms). Stedman's Medical Dictionary, Illustrated, (25th Ed.), Williams & Wilkins: Baltimore (1990) and Mosby's Medical, Nursing, & Allied Health Dictionary, (5th Ed.), Mosby: St. Louis (1998).

Any suitable antibiotic agent 16 may be employed, provided the antibiotic agent 16 effectively inhibits or prevents the growth, e.g., replication, or destroys the development of either Gram-positive or Gram-negative organisms and the antibiotic agent 16 remains stable in the second formulation 24. In one embodiment, the stability is over a prolonged period of time, e.g., up to about 2 years, up to about 1 year, or up to about 6 months, typically experienced in the manufacturing, packaging, shipping, and/or storage of the patch 1. Suitable antibiotic agents 16 are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; University of Wisconsin Antimicrobial Use Guide, http://www.medsch.wisc.edu/clinsci/amcg/amcg.html; Introduction on the Use of the Antibiotics Guideline, Descriptions of Specific Antibiotic Classes, Thomas Jefferson University, http://jeffline.tju.edu/CWIS/OAC/antibiotics_guide/intro.html; and references cited therein. The antibiotic agent 16 is useful in preventing and/or treating secondary infections that are typically encountered with viral infections.

Suitable antibiotic agents 16 include, but are not limited to, e.g., cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole, sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, ascariasis, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, triclosan, and pharmaceutically acceptable salts thereof.

Any suitable amount of antibiotic agent 16 may be employed, provided the amount of antibiotic agent 16 employed effectively inhibits or prevents the growth or destroys the development of either Gram-positive or Gram-negative organisms and the effective amount of the antibiotic agent 16 remains stable in the second formulation 24 over a prolonged period of time. Typically, the amount of antibiotic agent 16 depends upon the specific antibiotic agent 16 or agents employed. The antibiotic agent 16 may be present up to about 99.9 wt. % of the second formulation 24, up to about 50 wt. % of the second formulation 24, up to about 25 wt. % of the first formulation 5, or up to about 10 wt. % of the second formulation 24. In one embodiment, the antibiotic agent 7 may be present up to about 5.0 wt. % of the second formulation 24, up to about 1.0 wt. % of the second formulation 24, or up to about 0.5 wt. % of the second formulation 24.

Anti-Fungal Agent

The second formulation 24 may include one or more anti-fungal agents 23. Suitable anti-fungal agents 23 include, but are not limited to, e.g.,

[1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-Amino-3,6-dideoxy-β-D-mannopyranosypoxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid (Amphotericin B);

5-fluorocytosine (Flucytosine);

2,4-difluoro-α,α¹-bis(1H-1,2,4-triazol-1-ylmethyl)benzyl alcohol) (Fluconazole);

griseofulvin microsize (Griseofulvin);

(E)-N-(6,6-dimethyl-2-hepten-4-ynyl)-N-methyl-1-naphthalenemethanamine hydrochloride) (Terbinafine);

cis-1-acetyl-4-[4-[(2-(2,4-dichlorophenyl)-2-(1H-imadazol-1-ylmethyl)-1,3-dioxolan-4-yl]methoxy]phenyl]piperazine (Ketoconazole);

(∀)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methyoxy]phenyl]-1-piperazinyl]phenyl]-Δ²-1,2,4-triazolin-5-one mixture with (∀)-1-[(R*)-sec-butyl]-4-[p-[4-[p-[[(2S*,4R*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one or (∀)-1-[(RS)-sec-butyl]-4-[p-[4-[p-[[(2R,4S)-2-(2,4-dichlorophenyl)-2-(1H-1,2,4-triazol-1-ylmethyl)-1,3-dioxolan-4-yl]-methoxy]phenyl]-1-piperazinyl]phenyl]-Δ²-1,2,4-triazolin-5-one (Itraconazole);

2-chloro-5-hydroxy-1,3-dimethylbenzene (Chloroxylenol);

griseofulvin ultramicrosize (Griseofulvin);

(E)-N-(6,6,-dimethyl-2-hepten-4-ynyl)-N-methyl-1-naphthalenemanamine hydrochloride (Terbinafine);

6-cyclohexyl-1-hydroxy-4-methyl-2(1H)-pyridinone (Ciclopirox);

N-4-tert-butyl-benzyl-N-methyl-1-naphthalenemethylamine hydrochloride (Butenafine hydrochloride);

nystatin;

(E)-N-(Cinnamyl-N-methyl-1-naphthalenemethylamine hydrochloride (Naftifine hydrochloride);

1-(2,4-dichlorophenyl)-2-(1H-imidazol-1-yl)-O-[(2,4-dichlorophenypmethyl]oxime, (Z)-, mononitrate (Oxiconazole nitrate),

6-cyclohexyl-1-hydroxy-4-methyl-2(1H)-pyridone (Ciclopirox);

selenium sulfide;

(∀)-1-[4-(p-chlorophenyl)-2-[(2,6-dichlorophenypthio]butyl]imidazole mononitrate (Butoconazole nitrate);

1-(o-Chloro-α,α-diphenylbenzyl)imidazole (Clotrimazole);

(cis-1-[p-[[2-(2,4-dichlorophenyl)-2-(1H-1,2,4-triazol-1-ylmethyl)-1,3-dioxolan-4-yl]methoxy phenyl]-4-isopropyl-piperazine (Termayazole);

6-cyclohexyl-1-hydroxy-4-methyl-2(1H)-pyridone (ciclopirox);

5-chloro-2-(2,4-dichlorophenoxy)phenol (triclosan);

and combinations thereof.

The anti-fungal agent 23 may be present in the second formulation 24 in any suitable and appropriate amount. For example, the anti-fungal agent 23 may be present up to about 15 wt. %, up to about 10 wt. % or up to about 5 wt. % of the second formulation 24. Alternatively, the anti-fungal agent 23 may be present in the second formulation 24 in those amount as disclosed, e.g., in the Physician=s Desk Reference (PDR), 55^{th} edition (2001).

Antimicrobial Agent

The first formulation 5 and the second formulation 24 may each optionally include an antimicrobial agent 7 useful for preventing or inhibiting bacterial growth, mold growth, fermentation, and/or decomposition (e.g., preservative). As used herein, Aantimicrobial agent@ or Apreservative@ is any substance which prevents or inhibits bacterial growth, mold growth, fermentation, and/or decomposition. Concise Chemical and Technical Dictionary, 4th enlarged edition, Chemical Publishing Co., Inc., NY, N.Y. p. 939 (1986). Any suitable antimicrobial agent 7 may be employed, provided the antimicrobial agent 7 effectively prevents bacterial growth, mold growth, fermentation, and/or decomposition and the antimicrobial agent 7 remains stable in the first formulation 5 and/or the second formulation 24. In one embodiment, the stability is over a prolonged period of time, e.g., up to about 2 years, up to about 1 year, or up to about 6 months, typically experienced in the manufacturing, packaging, shipping, and/or storage of the patch 1.

Suitable antimicrobial agent 7 includes but is not limited to, e.g., quat-15, parabens, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, and pharmaceutically acceptable salts thereof. In one embodiment, the preservative is quat-15, which is commercially available from Dow Chemical (Midland Mich.).

The antimicrobial agent 7 may be employed in any suitable amount provided the amount of antimicrobial agent 7 effectively prevents or inhibits bacterial growth, mold growth, fermentation, and/or decomposition and the effective amount of antimicrobial agent 7 remains stable in the first formulation 5 and/or the second formulation 24 over a prolonged period of time. The antimicrobial agent 7 may be present in about 0.01 wt. % to about 99.9 wt. % of the first formulation 5 and/or the second formulation 24. The amount of antimicrobial agent 7 present in the first formulation 5 and/or the second formulation 24 typically depends upon the specific compound or compounds employed as the antimicrobial agent 7. For example, quat-15 may be employed in about 0.01 wt. % to about 1.5 wt. % of the first formulation 5 and/or the second formulation 24, in about 0.05 wt. % to about 0.15 wt. % of the first formulation 5 and/or the second formulation 24, or in about 0.08 wt. % to about 0.12 wt. % of the first formulation 5 and/or the second formulation 24.

Solvent

The solvent 13 may act as a carrier for, and in one embodiment, may dissolve, the antiviral agent 15; the antibiotic agent 16; the antifungal agent 23; the antimicrobial agent 7; and/or the adhesive 14. Any suitable solvent 13 may be employed, provided the solvent 13 effectively and independently dissolves the antiviral agent 15, the antibiotic agent 16, the antifungal agent 23, the antimicrobial agent 7, and/or the adhesive 14, and the solvent 13 remains stable in the second formulation 24 and the first formulation 5, if the solvent 13 is present in the first formulation 5. In one embodiment, the stability is over a prolonged period of time, e.g., up to about 2 years, up to about 1 year, or up to about 6 months, typically experienced in the manufacturing, packaging, shipping, and/or storage of the patch 1.

The solvent 13 may include one or more organic compounds, one or more inorganic compounds, or mixtures thereof. In one embodiment, the solvent 13 will include one or more organic compounds, e.g., esters, terpenes, alcohols, ketones, aldehydes, fatty acids, partially or fully esterified fatty acids, wherein the structures are cyclic, non cyclic (e.g., alkyl), alicyclic (e.g., a bridged ring compound) or aromatic, as well as organic compounds having combinations of these functional groups. Suitable exemplary solvents 13 are disclosed, e.g., in Aldrich Handbook of Fine Chemicals, 2000-2001 (Milwaukee, Wis.). In one embodiment, the solvent 13 includes water (e.g., deionized water).

In one embodiment of the present invention, the solvent 13 may include a (C₁-C₁₂)acyclic hydrocarbon, a (C₃-C₁₂)cyclic hydrocarbon, a (C₆-C₁₂)aryl hydrocarbon, a (C₆-C₁₂)heteroaryl hydrocarbon, or a (C₃-C₁₂)heterocyclic hydrocarbon;

wherein any of the hydrocarbons may optionally include one or more carbon-carbon double bonds and any of the hydrocarbons may optionally include one or more carbon-carbon triple bonds;

wherein any of the hydrocarbons may optionally include one or more oxy (—O—), carbonyl(—C(═O)C—), carboxylato(—C(═O)O—), dioxy(—O—O—), dithio(—S—S—), imino(—NH—), methylene dioxy(—OCH₂O—), sulfinyl(—SO—), sulfonyl(—SO₂—), or thio(—S—);

wherein any of the hydrocarbons may optionally be substituted with one or more amino, hydroxyl, cyano, nitro, (C₁-C₁₂)alkoxy, halo, trifluoro, trifluoro (C₁-C₁₂)alkyl, NR¹R², or COOR¹; wherein R¹ and R² are each independently hydrogen, a (C₁-C₁₂)acyclic hydrocarbon or a (C₃-C₁₂)cyclic hydrocarbon.

The composition of the solvent 13 in the first formulation 5 may be the same as the composition of the solvent 13 in the second formulation. Alternatively, the composition of the solvent 13 in the first formulation 5 may be different from the composition of the solvent 13 in the second formulation 24.

The solvent 13 may be employed in any suitable amount, provided the amount of solvent 13 is effective to independently dissolve the antiviral agent 15, the antibiotic agent 16, the antifungal agent 23; the antimicrobial agent 7, and/or the adhesive 14 and the effective amount of solvent 13 remains stable in the first formulation 5 and the second formulation 24. In one embodiment, the stability is over a prolonged period of time, e.g., up to about 2 years, up to about 1 year, or up to about 6 months, typically experienced in the manufacturing, packaging, shipping, and/or storage of the patch 1.

In one embodiment, one or more fragrances 8 may be employed as a solvent 13. In another embodiment, one or more fragrances 8 may be employed in addition to the use of a solvent 13. As used herein, a Afragrance@ is a compound that emits a sweet or pleasant odor or scent. The Amerimay Heritage Dictionary of the English Language, Houghton Mifflin Company, Boston, Mass., p. 521 (1981). The fragrance 8 may either at least partially mask the odor of the antiviral agent 15, the antibiotic agent 16, the antifungal agent 23, and/or the antimicrobial agent 7, if such an odor is present, or the fragrance 8 may provide a pleasant odor to the patch 1. In one embodiment, the pleasant odor may be a floral scent, a food scent, a fruit scent, a plant leaf scent, or any combination thereof. As such, the odor of the patch 1 may be pleasant to the patient, due to the odor or scent of the one or more fragrances.

A suitable fragrance 8 may be employed as a carrier (e.g., solvent) or co-carrier (e.g., co-solvent). The use of a fragrance 8 such as eucalyptus oil as a solvent 13 requires lower amounts of total solvent 13 to be used. Lower amounts of total solvent 13 may allow for improved manufacturing characteristics. For example, if no fragrance 8 (e.g., eucalyptus oil) is employed as a solvent 13, about 8.0 wt. % propylene glycol may be needed to dissolve the antiviral agent 15, antibiotic agent 16, and/or antifungal agent 23. The use of a suitable fragrance 8 (e.g., eucalyptus oil) may result in only about 1.6 wt. % of the fragrance 8 and only about 2.0 wt. % of propylene glycol used to dissolve the antiviral agent 15, antibiotic agent 16, and/or antifungal agent 23 (e.g., camphor and lidocaine). As such, less overall amounts of solvent 13 may be required when a fragrance 8 such as eucalyptus oil is employed. The use of lower amounts of glycerin, ethylene glycol, and/or propylene glycol may allow the adhesive patch 1 to be more effectively coated during the manufacturing process. In one embodiment, the use of lower amounts of propylene glycol may result in less bleed-through or leak-through of the second formulation 24 onto the back side 4 of backing 2 in manufacturing of the adhesive patch 1. As such, the use of a suitable fragrance 8 as a solvent 13 may result in less bleed-through or leak-through of the second formulation 24 onto the back side 4 of backing 2 in manufacturing of the adhesive patch 1.

In one embodiment, the suitable fragrance 8 is a non-irritant to mammalian (e.g., human) skin. As used herein, “non-irritant” refers to an agent, e.g., organic compound, that does not produce an appreciable or significant amount of inflammation or irritation when applied topically to the skin of a mammal in the specified amount. As such, in one embodiment, the fragrance 8 is one that is pharmaceutically acceptable for topical use.

In one embodiment, the fragrance 8 has a low to moderate volatility, so that its evaporation from the patch 1 is rendered minimal to moderate. The volatility may, however, be high enough such that when desirable, the odor or scent may be detected by the patient. In one embodiment, the second formulation 24 of the adhesive patch 1 may emit an odor or scent, due to the fragrance 8, that is detected by the patient for a period of at least about 10 hours, at least about 8 hours, or at least about 6 hours.

Any suitable fragrance 8 may be employed, provided the fragrance 8 effectively dissolves the antiviral agent 15, the antibiotic agent 16, the antifungal agent 23, the antimicrobial agent 7, and/or the adhesive 14; the fragrance 8 remains stable in the second formulation 24; and the fragrance 8 either at least partially masks the odor of the antiviral agent 15, the antibiotic agent 16, the antifungal agent 23, and/or the antimicrobial agent 7, if such an odor is present, or provides a pleasant odor to the patch 1. In one embodiment, the stability is over a prolonged period of time, e.g., up to about 2 years, up to about 1 year, or up to about 6 months, typically experienced in the manufacturing, packaging, shipping, and/or storage of the patch 1. It is appreciated that the suitable fragrances would be known to those skilled in the art. It is also appreciated that those skilled in the art understand that suitable fragrances are commercially available from, for example, Alpine Aromatics (Piscataway, N.J.), Andrea Aromatics (Princeton, N.J.), Arylessence, Inc. (Marietta, Ga.), Belmay Co., Inc. (Yonkers, N.Y.), Crami Flavor & Fragrance Co., Inc. (City of Commerce, Calif.), Creative Fragrances Mfgr. Inc. (Dallas, Tex.), Drom International Co. (Tawaco, N.J.), Fleurchem, Inc. (Middletown, N.Y.), Great Lakes Chem. Corp. (Lafayette, Ind.), Kraus & Co., Inc. (Battle Creek, Mich.), The Lebermuth Co., Inc. (Mishawaka, Ind.), Penta Manufacturing (Livingston, N.J.), Shaw Mudge & Co. (Shelton, Conn.), Synarome Corp. (NY, N.Y.), Penreco (Houston, Tex.), Tracy Chemical Co. (Portland, Oreg.), Belle-Aire Fragrances (Mundelein, Ill.), Gusta Fragrances Co. (Chesire, Conn.), Atlanta Fragrance (Kennesaw, Ga.), and Bell Flavors & Fragrances, Inc (Northbrook, Ill.).

As the number of suitable fragrances is too voluminous and expansive to exhaustively list herein, suitable exemplary fragrances are disclosed herein. Suitable exemplary fragrances include but are not limited to grape fragrance, musk fragrance, light vanilla fragrance, Jergens lotion fragrance, Vaseline Intensive Care fragrance, Nivea Lotion fragrance, Ivory Soap fragrance, amaretto, blueberry, coffee, egg nog, peanut butter, rum cake, honey almond, ginger bread house, coffee cake & spice, raspberry rose, sassafras, strawberry, grapefruit pink, home sweet, jeweled citrus, lemon, mango, mulberry, orange flower, passion fruit, pikaki, freesia, china rain, coconut, apple, baked bread, cornucopia, lemon chiffon, peppermint twist, white cake, cherry pie, sugar plum, plum, romantic, sea fresh, tea, green floral, honeydew, kiwi, lilac, may bouquet, neutralizer, patchouli, peach, pine apple blossom, chocolate mint, frankincense, baked apple pie, cappuccino, cran-apple, maple syrup, popcorn (buttered), sugar cookie, cotton maydy, cranberry cobbler, plumeria, rum, spring fever, watermelon, guava, honeysuckle, hyacinth, macadamia nut, melon, oakmoss, papaya, pear pineapple, blueberry, citrus-ginseng, garden dreams, banana creme pie, chocolate mint, cranberry, macadamia nut, pumpkin pie, chocolate German cake, banana nut bread, sweet potato pie, raspberry, sandalwood, spring flowers, ylang, heather, jasmine, lavender, magnolia, mountain air, orange essence, paradise, peony, alpine breeze, chamomile, clover, gardenia, or any combination thereof. In one embodiment, the fragrance 8 is eucalyptus oil.

Any suitable amount of fragrance 8 may be employed, provided the effective amount of fragrance 8 effectively dissolves the antiviral agent 15, the antibiotic agent 16, the antifungal agent 23, the antibiotic agent 16, the antimicrobial agent 7, and/or the adhesive 14; the effective amount of fragrance 8 remains stable in the first formulation 5; and the effective amount of fragrance 8 either at least partially masks the odor of the antiviral agent 15, the antibiotic agent 16, the antimicrobial agent 7 and/or the antifungal agent 23, if such an odor is present, or provides a pleasant odor to the patch 1 over a prolonged period of time. The suitable amount of fragrance 8 may depend upon the specific fragrance 8 or fragrances 8 employed.

When the adhesive patch 1 of the present invention is used with patients having received a vaccination (e.g., cowpox vaccination), the solvent 13 may optionally not include an alcohol or any other substance that would kill or weaken a live virus, e.g., vaccinia virus. Specifically, when the adhesive patch 1 of the present invention is used with patients having received a vaccination (e.g., cowpox vaccination), the solvent 13 optionally does not include (C₁-C₁₂)alkyl or (C₃-C₁₂)cycloalkyl substituted with one or more hydroxyl groups. More specifically, when the adhesive patch 1 of the present invention is used with patients having received a vaccination (e.g., cowpox vaccination), the solvent 13 optionally does not include isopropyl alcohol.

Essential Oil

As used herein, an Aessential oil@ 21 refers to a highly odoriferous, volatile liquid component obtained from plant tissue. Essential oils 21 typically include a mixture of one or more terpenes, esters, aldehydes, ketones, alcohols, phenols, and/or oxides. These functional classes of compounds are responsible for the therapeutic properties and distinct fragrance of the essential oil.

In one embodiment, the essential oil 21 is not: methyl salicylate, menthol, camphor, eucalyptus oil, spearmint oil, or a combination thereof.

In one embodiment of the present invention, the second formulation 24 may include methyl salicylate, menthol, camphor, eucalyptus oil, spearmint oil, or a combination thereof. In such an embodiment, the second formulation 24 may also include one or more essential oils 21 as defined herein.

In one embodiment of the present invention, the essential oil 21 is not: oil of wintergreen, thymol, oil of peppermint, spirits of turpentine, ephedra, coltsfoot, ginger, cinnamon oil, fir needle oil, lemon oil, Peruvian Balsam, or a combination thereof. Again, in such an embodiment, the second formulation 24 of the present invention may include any one or more of oil of wintergreen, thymol, oil of peppermint, spirits of turpentine, ephedra, coltsfoot, ginger, cinnamon oil, fir needle oil, lemon oil, Peruvian Balsam, or a combination thereof; provided an essential oil 21 as defined herein is included in the second formulation 24.

In one embodiment of the present invention, the second formulation 24 may include oil of wintergreen, thymol, oil of peppermint, spirits of turpentine, ephedra, coltsfoot, ginger, cinnamon oil, fir needle oil, lemon oil, Peruvian Balsam, or a combination thereof. In such an embodiment, the second formulation 24 may also include one or more essential oils 21 as defined herein.

The essential oil 21 may be manufactured (i.e., synthesized or partially synthesized). Alternatively, the essential oil 21 may be obtained from a plant or plant component (e.g., plant tissue). Suitable plant or plant components include, e.g., a herb, flower, fruit, seed, bark, stem, root, needle, bulb, berry, rhizome, rootstock, leaf, or a combination thereof.

Any suitable essential oil 21 may be employed provided (1) the essential oil 21 has therapeutic properties (e.g., the essential oil 21 effectively relieves discomfort), (2) the essential oil 21 provides a scent that is associated with plant tissue, and/or (3) the essential oil 21 remains stable in the second formulation 24. In one embodiment, the stability is over a prolonged period of time, e.g., up to about 3 years, up to about 1 year, or up to about 6 months, typically experienced in the manufacturing, packaging, shipping, and/or storage of the adhesive skin patch 1. The specific essential oil 21 may be non-toxic to mammals (e.g., humans) and may be suitable for medicinal use (e.g., topically or via inhalation). The specific essential oil 21 may also comply with any controlling or governing body of law, e.g., FDA regulations.

Suitable specific essential oils 21 include but are not limited to, e.g., one or more of the following: ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, maynabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Morocmay chamomile, Morocmay wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenugreek, fir, Mayada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaimay gold, unrefined Jamaimay gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, Arectified® melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, Indian patchouli oil, Indonesian patchouli oil, peanut, peanut oil, pemay oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce, St. John=s wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra.

In one embodiment, suitable exemplary essential oils 21 include, e.g., angelica root, anise, basil (e.g., sweet French basil), bay leaf, benzoin absolute, bergamot, birch, carrot seed, cedarwood, chamomile (e.g., German chamomile, Morocmay chamomile, or Roman chamomile), cinnamon leaf, cinnamon cassia, cistus, citronella, clary sage, clove bud, cypress, eucalyptus globulus, eucalyptus citriodora, everlasting (helicrysum), fennel, fir, frankincense, geranium, ginger, grapefruit, helichrysum, hyssop, juniper berry, lavender, lavendin, lemon, lemongrass, lime, marjoram, myrrh, myrtle, neroli, niaouli, nutmeg, sweet orange, oregano, patchouli, pennyroyal, peppermint, petitgrain, pepper, pine needle, ravensare, rose geranium, rosemary (e.g., Spanish rosemary), rosewood, sage, sandalwood, spikenard, spruce, tangerine, tarragon, tea tree, thyme, vanilla, vetiver, ylang ylang, or a combination thereof.

Other suitable essential oils 21 that may be employed in the adhesive skin patch 1 of the present invention are disclosed in the accompanying documents herein, which form part of this patent application. Other suitable essential oils 21 that may be employed in the adhesive skin patch 1 of the present invention are disclosed in the following websites: www.essential-essences.com; www.fragrancefactory.com; www.essentialoil.com; www.essentialoils.org; www.halcyon.com; and www.essential-oil.org; which are all incorporated by reference herein.

The essential oil 21 may be present in any appropriate and suitable amount, provided (1) the amount of essential oil 21 has therapeutic properties (e.g., the amount of essential oil 21 effectively relieves discomfort), (2) the amount of essential oil 21 provides a scent that is associated with plant tissue, and/or (3) the amount of essential oil 21 remains stable in the second formulation 24. In one embodiment, the stability is over a prolonged period of time, e.g., up to about 3 years, up to about 1 year, or up to about 6 months, typically experienced in the manufacturing, packaging, shipping, and/or storage of the adhesive skin patch 1. The specific amount of essential oil 21 may be non-toxic to mammals (e.g., humans) and may be suitable for medicinal use (e.g., topically or via inhalation). The specific amount of essential oil 21 may also comply with any controlling or governing body of law, e.g., FDA regulations.

Typically, the amount of essential oil 21 present in the second formulation 24 depends upon the specific compound or compounds employed as the essential oil 21. In one embodiment, the essential oil 21 may be present in about 0.01 wt. % to about 99.9 wt. % of the formulation 5. In one embodiment, the essential oil 21 may be present up to about 50 wt. % of the second formulation 24, up to about 25 wt. % of the second formulation 24, up to about 20 wt. % of the second formulation 24, up to about 10 wt. % of the second formulation 24, or up to about 5 wt. % of the second formulation 24.

In one embodiment of the present invention, angelica root, anise, basil (e.g., sweet French basil), bay leaf, benzoin absolute, bergamot, birch, carrot seed, cedarwood, chamomile (e.g., German chamomile, Morocmay chamomile, or Roman chamomile), cinnamon leaf, cinnamon cassia, cistus, citronella, clary sage, clove bud, cypress, eucalyptus globulus, eucalyptus citriodora, everlasting (helicrysum), fennel, fir, frankincense, geranium, ginger, grapefruit, helichrysum, hyssop, juniper berry, lavender, lavendin, lemon, lemongrass, lime, marjoram, myrrh, myrtle, neroli, niaouli, nutmeg, sweet orange, oregano, patchouli, pennyroyal, peppermint, petitgrain, pepper, pine needle, ravensare, rose geranium, rosemary (e.g., Spanish rosemary), rosewood, sage, sandalwood, spikenard, spruce, tangerine, tarragon, tea tree, thyme, vanilla, vetiver, ylang ylang, or a combination thereof, or any combination thereof, may be present up to about 20 wt. % of the formulation, up to about 10 wt. % of the formulation, or up to about 5 wt. % of the formulation.

The adhesive skin patch 1 may include an essential oil 21 located in at least a portion of the back side 4 of the backing 2, on at least a portion of the back side 4 of the backing 2, or on and in at least a portion of the back side 4 of the backing 2. As such, the essential oil 21 may be located on the entire surface of the back side 4 of the backing 2 or the essential oil 21 may be located on a portion of the surface of the back side 4 of the backing 2. In one embodiment, the essential oil 21 may be located on the entire surface of the back side 4 of the backing 2.

In addition to being located on the surface of the back side 4 of the backing 2, the essential oil 21 may be located in at least a portion of the underlying surface of the back side 4 of the backing 2 (e.g., the essential oil 21 may be partially embedded into the backing 2). As shown in FIGS. 7 and 8, the essential oil 21 may penetrate a substantial portion of the back side 4 of the backing 2, as disclosed, e.g., in U.S. Pat. No. 5,536,263, and references cited therein. For example, the essential oil 21 may penetrate about one-tenth to about nine-tenths the thickness of the backing 2, or about one-fourth to about nine-tenths the thickness of the backing 2. As such, the essential oil 21 may be partially embedded into the backing 2.

In one embodiment, the essential oil 21 may be located on the entire back side 4 of the backing 2 and partially in the back side 4 of the backing 2 (e.g., the essential oil 21 is partially embedded into the backing 2). Alternatively, a portion of the back side 4 of the backing 2 may include the essential oil 21.

Plant Tissue

The essential oil 21 may be derived from plant tissue.

As used herein, “plant tissue” refers to the tissue of any organism of the plant kingdom, as opposed to one of the animal kingdom or of the kingdoms of Fungi, Protista, or Monera. The plant tissue may be any portion or portions of the plant (e.g., bark, roots, leaves, flowers, needles, bulbs, berries, rhizomes, rootstocks, stems, and seeds), as well as the entire plant. The tissues of a plant (“plant tissue”) generally fall into three main categories: dermal tissue, ground tissue, and vascular tissue. Dermal tissue refers to the “skin” layer of all plant organs and is responsible for environmental interaction (light passage, gas exchange, pathogen recognition and protection, color display, etc.). Dermal tissue is composed of epidermal cells, closely packed cells that secrete a waxy cuticle that aids in the prevention of water loss. Ground tissue lies between dermal tissue and vascular tissue. The ground tissue comprises the bulk of the primary plant body. Parenchyma, collenchyma, and sclerenchyma cells are common in the ground tissue. In roots, the ground tissue may store sugars or starches to fuel the spring sap flow; in leaves, the ground tissue is the layer responsible for photosynthesis (the mesophyll). Vascular tissue transports food, water, hormones and minerals within the plant. Vascular tissue includes xylem, phloem, parenchyma, and cambium cells.

As used herein, “bark” refers to the dry, dead outer covering of woody branches, stems and roots of plants that is very distinct and separable from the wood itself. It includes all tissue outside the cambium (growth layer between bark and wood).

As used here the terms “leaf' or “leaves” refer to those parts of a plant which grow along the sides of branches or stems or at the bases of plants. Most are green and contain chlorophyll, though they vary in their shapes and sizes. Leaves are the part of the plant that ordinarily performs photosynthesis (the process that converts sunlight and carbon dioxide into energy).

As used herein, “needle” generally refers to a narrow stiff leaf, such as those of conifers (e.g., pine trees).

As used herein, “root” refers to the part of a plant, normally underground, that absorbs nutrients and anchors the plant into the ground.

As used herein, “bulb” refers to a spheroidal body growing from a plant either above or below the ground (usually below), which is usually a bud, consisting of a cluster of partially developed leaves, and producing, as it grows, a stem above, and roots below, (e.g., the onion or tulip bulb). A true bulb is a complete package containing next year's plant (flower) already forming inside. The contents of the bulb are often enclosed in protective, fleshy scales, which are held together by a small basal plate. The scales are modified leaves that contain enough nutrients to sustain the plant through dormancy and early growth. They may be loose and open like those of a lily, or tightly closed like those of a hyacinth. In many bulbs, a paper-thin tunic protects the scales (lilies don't have a tunic). Roots will grow from the bulb's basal plate.

As used herein, “berry” refers to any small fruit that is pulpy or succulent throughout, having seeds loosely imbedded in the pulp, such as the currant, grape, or blueberry. Berry may be further defined as an indehiscent fruit derived from a single ovary and having the whole wall fleshy, such as the grape or tomato. Furthermore, berries come in various structures including simple, such as grape; blueberry, cranberry, or aggregate, such as blackberry; raspberry, strawberry mulberry.

As used herein, “rhizome” refers to a horizontal, usually underground stem that often sends out roots and shoots from its nodes (also called rootstalk or rootstock).

As used herein, “rootstock” refers to a robust plant that provides the root system in grafting, also known as a stock. Scions and buds are grafted and budded to a rootstock or stock. Rootstock also refers to the elongated and often thick rhizomes of certain perennial herbaceous plants such as the Iris, Aspidistra and Solomon's Seal.

As used herein, “stem” refers to the main (usually aerial) axis (sometimes referred to as the trunk or stalk) of a tree, shrub, or plant. “Stem” also refers to the part of the plant that supports the leaves, flowers or fruits of a plant, such as the peduncle of a fruit or the pedicel of a flower.

As used herein, “seed” refers to a ripened ovule, consisting of an embryo with one or more integuments, or coverings, such as an apple seed, a currant seed, dill seed, or kola nut seed. By germination, most seeds produce a new plant. “Seed” also refers to any small seedlike fruit, though it may consist of a pericarp, or even a calyx, as well as the seed proper, such as a parsnip seed or thistle seed. The seed proper has an outer and an inner coat, and within these the kernel or nucleus. The kernel is either the embryo alone, or the embryo enclosed in the albumen, which is the material for the nourishment of the developing embryo. The scar on a seed, left where the stem parted from it, is called the hilum, and the closed orifice of the ovule, the micropyle.

In one embodiment, the solvent 13 may be a fragrance 8 that may either at least partially mask the odor of the antiviral agent 15, the antibiotic agent 16, the antifungal agent 23, and/or the antimicrobial agent useful for preventing or inhibiting bacterial growth, mold growth, fermentation, and/or decomposition 7, if such odor is present; or may provide a pleasant odor to the patch 1.

Topically Suitable Oil

Any topically suitable oil 27 may be employed, provided that the topically suitable oil 27 helps to dissolve the antiviral agent 15, the antibiotic agent 16, and/or the antifungal agent 23; keeps the antiviral agent 15, the antibiotic agent 16, and/or the antifungal agent 23 soluble over time; and prevents the antiviral agent 15, the antibiotic agent 16, and/or the antifungal agent 23 from evaporating over the period of use. The topically suitable oil 27 is generally recognized as safe (GRAS) for topical use. Topically suitable oils 27 are described, for example, in the Handbook of Pharmaceutical Excipients, Pharmaceutical Press, (5^{th} ed.), 2006. The topically suitable oil 27 may be used in any appropriate and suitable amount from 1 to 90 wt. % of the second formulation 24. In one embodiment, the topically suitable oil 27 may be 10-30 wt. % or 30-80 wt. % of the second formulation 24. The topically suitable oil 27 is compatible with the adhesive patch 1.

Adhesive

Any suitable adhesive 14 may be employed, provided the adhesive 14 provides the requisite adhesiveness to the patch 1 and the adhesive 14 remains stable in the first formulation 5. In one embodiment, the stability is over a prolonged period of time, e.g., up to about 2 years, up to about 1 year, or up to about 6 months, typically experienced in the manufacturing, packaging, shipping, and/or storage of the patch 1. It is appreciated that the suitable adhesives would be known to those skilled in the art. Suitable adhesives are disclosed, e.g., in U.S. Pat. No. 4,675,009; U.S. Pat. No. 5,536,263; U.S. Pat. No. 4,696,854; U.S. Pat. No. 5,741,510, and references cited therein. In one embodiment the adhesive 14 is an acrylic ester copolymer.

Any suitable amount of adhesive 14 may be employed, provided the amount of adhesive 14 effectively provides the requisite adhesiveness to the patch 1 and the effective amount of the adhesive 14 remains stable in the first formulation 5 over a prolonged period of time. Typically, the suitable amount of adhesive 14 depends upon the specific adhesive 14 or adhesives 14 employed. The first formulation 5 may include an adhesive 14 in about 0.1 wt. % to about 50 wt. % of the first formulation 5. In one embodiment, the first formulation 5 may include an adhesive 14 in about 0.5 wt. % to about 10.0 wt. % of the first formulation 5. In one embodiment, the first formulation 5 may include an adhesive 14 in about 1.0 wt. % to about 15.0 wt. % of the first formulation 5.

Alternatively, the adhesive 14 may include a hot melt pressure sensitive adhesive or solvent based pressure sensitive adhesive (e.g., polyacrylate, polyisobutylene, and polybutene), rubber, silicone based pressure sensitive adhesives (e.g., polydimethylsiloxane and resin mixtures), polystyrene-polybutadiene-polystyrene, polystyrene-polyisoprene-polystyrene, polystyrene-poly(ethylene-butylene)-polystyrene block polymers, or any combination thereof. In addition, the adhesive 14 may include a resin emulsion adhesive, wherein the resin emulsion adhesive may include vinyl acetate resin, acrylic ester copolymer, vinyl actetate/diocyl maleate copolymer, acrylic copolymer, or any combination thereof.

Other suitable adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein.

The adhesive 14 may be located on and in any portion of the first formulation 5. In one embodiment, the adhesive 14 may be located on the entire skin contact side of the first formulation 5. When the adhesive skin patch 1 is placed upon the skin surface of a patient, the adhesive 14 in this configuration is in continuous contact with the skin surface of the patient.

Polymers

The first formulation 5 and/or second formulation 24 may optionally include one or more polymers 9. The polymer 9 provides structure and strength to the adhesive 14. Any suitable polymer 9 may be employed, provided the polymer 9 provides structure and strength to the adhesive 14 and the polymer 9 remains stable the first formulation 5. In one embodiment, the stability is over a prolonged period of time, e.g., up to about 2 years, up to about 1 year, or up to about 6 months, typically experienced in the manufacturing, packaging, shipping, and/or storage of the patch 1. Suitable polymers 9 include, e.g., starch, starch derivatives, polyvinyl pyrrolidone, polyethylene oxide, polyacrylate quats, polymaleic acid, polymaleic anhydride, polyurethanes, polyureas, karaya, gum acacia, locust bean gum, xanthan gum, guar gum, modified guar gum, maltodextrin, carboxymethyl cellulose, carboxypropyl cellulose, polyacrylamide, polyvinyl alcohol, poly AMPS, and polyacrylates. Other suitable polymers 9 are disclosed, e.g., in U.S. Pat. No. 4,675,009; U.S. Pat. No. 5,536,263; U.S. Pat. No. 4,696,854; U.S. Pat. No. 5,741,510, and references cited therein. In one embodiment, the polymer 9 is karaya.

Any suitable amount of polymer 9 may be employed, provided the amount of polymer 9 effectively provides structure and strength to the adhesive 14 and the effective amount of polymer 9 remains stable the first formulation 5 over a prolonged period of time. The suitable amount of polymer 9 may depend upon the specific polymer 9 or polymers 9 employed. For example, karaya may be employed as the polymer 9 in about 10 wt % to about 55 wt. % of the first formulation 5 and/or second formulation 24, in about 20 wt % to about 35 wt. % of the first formulation 5 and/or second formulation 24, or in about 23 wt % to about 29 wt. % of the first formulation 5 and/or second formulation 24. In one embodiment, karaya may be employed as the polymer 9 in about 24 wt % to about 28 wt. % of the first formulation 5 and/or second formulation 24.

Humectant

The first formulation 5 and/or second formulation 24 may optionally include one or more humectants 17 to provide a moistening effect to the adhesive 14. For example, the humectant 17 may hydrate the polymer 9. Any suitable humectant 17 may be employed, provided the humectant 17 effectively provides a moistening effect to the adhesive 14 and the humectant 17 remains stable in the first formulation 5. In one embodiment, the stability is over a prolonged period of time, e.g., up to about 2 years, up to about 1 year, or up to about 6 months, typically experienced in the manufacturing, packaging, shipping, and/or storage of the patch 1. One suitable humectant 17 is glycerin. Other suitable humectants 17 s include polyhydric alcohols such as ethylene glycol, propylene glycol, triethylene glycol, tetraethylene glycol, and sorbitol.

Any suitable amount of humectant 17 may be employed, provided the amount of humectant 17 effectively provides a moistening effect to the adhesive 14 and the effective amount of humectant 17 remains stable in the first formulation 5. The suitable amount of humectant 17 may depend upon the specific humectant 17 or humectants 17 employed and the specific polymer 9 or polymers 9 employed. For example, karaya may be employed as the polymer 9 and glycerin may be employed as the humectant 17 in about 20 wt % to about 70 wt. % of the first formulation 5 and/or second formulation 24, and in one embodiment about 30 wt % to about 60 wt. % of the first formulation 5 and/or second formulation 24, or about 40 wt % to about 50 wt. % of the first formulation 5 and/or second formulation 24.

Topical Moisturizer

The first formulation 5 and/or second formulation 24 may optionally include a topical moisturizer 18 (e.g., skin protectant). Any suitable topical skin protectant may be employed, provided the skin is effectively protected or moisturized and the skin protectant remains stable in the first formulation 5 and/or second formulation 24. In one embodiment, the stability is over a prolonged period of time, e.g., up to about 2 years, up to about 1 year, or up to about 6 months, typically experienced in the manufacturing, packaging, shipping, and/or storage of the patch 1. Suitable skin protectants include, e.g. aloe, lanolin, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, trolamine, white petrolatum, zinc acetate, zinc carbonate zinc oxide, zinc sulfate, shea butter, and any combination thereof.

As used herein, Acalamine@ is a pink powder of zinc oxide and a skin protectant containing about 98% zinc oxide and about 0.5% ferric oxide; Aaloe@ is the dried latex of leaves of Curaco Aloe (Aloe barbadenis Miller, Aloe vera Linne) or Cape Aloe (Aloe ferox Miller and hybrids), of the family Liliacaea; AVitamin E@ is 3,4-dihydro-2,5,7,8-tetramethyl-2-(4,8,12-trimethyltridecyl)-2H-1-benzopyran-6-ol; AVitamin E acetate@ is 3,4-dihydro-2,5,7,8-tetramethyl-2-(4,8,12-trimethyltridecyl)-2H-1-benzopyran-6-ol acetate; and Alanolin@ is the fat-like secretion of the sebaceous glands of sheep (i.e., complex mixture of esters and polyesters of 33 high molecular weight alcohols and 36 fatty acids) which is deposited onto the wool fibers. In one embodiment, the topical moisturizer 18 may be aloe and Vitamin E.

Aloe is commercially available as Aloe Vera•Gel from Terry Laboratories (Melbourne, Fla.). Aloe Vera Gel is commercially available as Aloe Vera Gel 40× (20.0 wt. % solution in water), Aloe Vera Gel 1× (0.5 wt. % solution in water), Aloe Vera Gel 10× (5.0 wt. % solution in water), or solid Aloe Vera. The solid Aloe Vera may be dissolved in a carrier, such as water, to the desired concentration. In addition, the commercially available forms of Aloe Vera are optionally available as decolorized Aloe Vera.

Any suitable amount of topical moisturizer 18 may be employed, provided the suitable amount of skin protectant effectively protects or moisturizes the skin and the effective amount of skin protectant remains stable in the first formulation 5 and/or second formulation 24 over a prolonged period of time. The suitable and effective amount of topical moisturizer 18 may depend in part upon the specific moisturizer 18 or moisturizers 18 present in the first formulation 5 and/or second formulation 24. For example, Aloe Vera Gel, 10× may be present up to about 40.0 wt. % of the first formulation 5 and/or second formulation 24. In one embodiment, Aloe Vera Gel, 10× may be present up to about 5.0 wt. % of the first formulation 5 and/or second formulation 24. In one embodiment, Aloe Vera Gel, 10× may be present up to about 1.0 wt. % of the first formulation 5 and/or second formulation 24. In addition, Vitamin E acetate may be present up to about 5 wt. % of the first formulation 5 and/or second formulation 24. In one embodiment, Vitamin E acetate may be present up to about 1.0 wt. % of the first formulation 5 and/or second formulation 24. In one embodiment, Vitamin E acetate may be present up to about 0.5 wt. % of the first formulation 5 and/or second formulation 24.

Polyhydric Alcohol

The second formulation 24 optionally may include one or more polyhydric alcohols 22. Suitable polyhydric alcohols 22 include, e.g., ethylene glycol, propylene glycol, triethylene glycol, tetraethylene glycol, sorbitol, or any combination thereof. Specifically, the polyhydric alcohol 22 may include propylene glycol.

Any suitable amount of polyhydric alcohol 22 may be employed. For example, the polyhydric alcohol 22 may be present up to about 35 wt. % of the second formulation 24, up to about 15 wt. % of the second formulation 24, or up to about 5 wt. % of the second formulation 24. In one embodiment, the polyhydric alcohol 22 may be present in about 0.5 wt. % to about 5.0 wt. % of the second formulation 24.

Water

The first formulation 5 and/or second formulation 24 may optionally include water, e.g., deionized water (DI). Any suitable amount of water may be employed, provided the amount of water maintains the adhesiveness of the adhesive 14 and maintains the appropriate stability of the first formulation 5 and/or second formulation 24. For example, deionized water may be present up to about 50 wt. % of the first formulation 5 and/or second formulation 24, up to about 40.0 wt. % of the first formulation 5 and/or second formulation 24, or up to about 30.0 wt. % of the first formulation 5 and/or second formulation 24. In one embodiment, deionized water may be present up to about 20.0 wt. % of the first formulation 5 and/or second formulation 24. In one embodiment, deionized water may be present up to about 10.0 wt. % of the first formulation 5 and/or second formulation 24. In one embodiment, deionized water may be present in about 5.0 wt. % to about 15.0 wt. % of the first formulation 5 and/or second formulation 24.

The adhesive skin patch 1 may have any suitable size and shape. In addition, the adhesive skin patch 1 may be cut, as desired, to provide an adhesive skin patch 1 of a suitable size and shape. The adhesive skin patch 1 may be cut with any suitable cutting device such as a scissors, scalpel, or knife.

In one embodiment, the adhesive skin patch 1 has a length of about 0.1 inch to about 12 inches, about 0.1 inch to about 8 inches, of about 0.20 inch to about 4 inches, or about 0.2 inches to about 2.0 inches. In one embodiment, the adhesive skin patch 1 has a length of about 1.0 inch to about 8 inches, about 2 inches to about 6 inches, or about 3 inches to about 4 inches.

In one embodiment, the adhesive skin patch 1 has a width of about 0.1 inch to about 12.0 inches, about 0.1 inch to about 4 inches, about 0.20 inches to about 2.0 inches, or about 0.2 inches to about 1.0 inch. In one embodiment, the adhesive skin patch 1 has a width of about 1.0 inch to about 8 inches, about 2 inches to about 6 inches, or about 3 inches to about 4 inches.

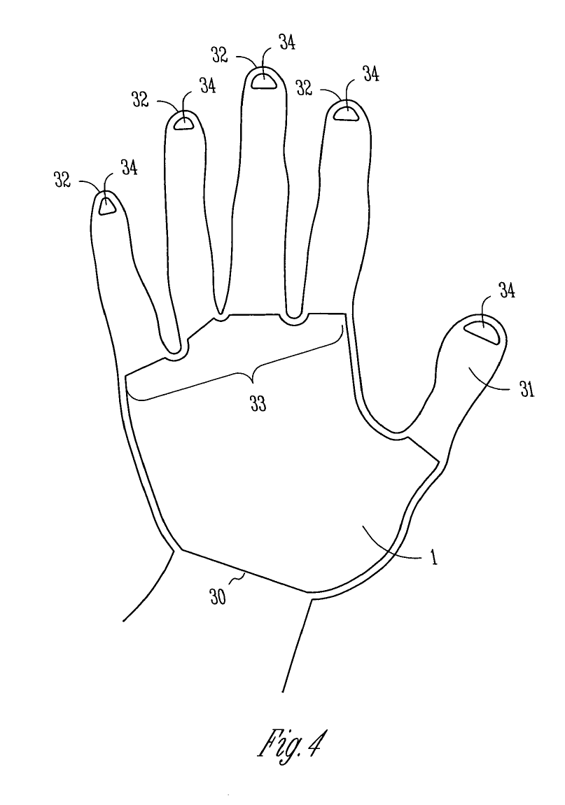

In one specific embodiment of the present invention, the adhesive skin patch 1 may be oval or elliptical in shape (see, FIG. 3). The oval or elliptical patch 1 may have a length of about 0.25 inches to about 0.50 inches and a width of about 0.25 inches to about 0.50 inches. See, FIG. 3. In another specific embodiment of the present invention, the adhesive skin patch 1 may have a circular shape. The circular patch 1 may have a diameter of about 0.25 inches to about 0.50 inches.

In another specific embodiment of the present invention, the adhesive skin patch 1 may be in the shape of the dorsal portion of a hand between the wrist 30 and the knuckles 33 (see, FIG. 4).

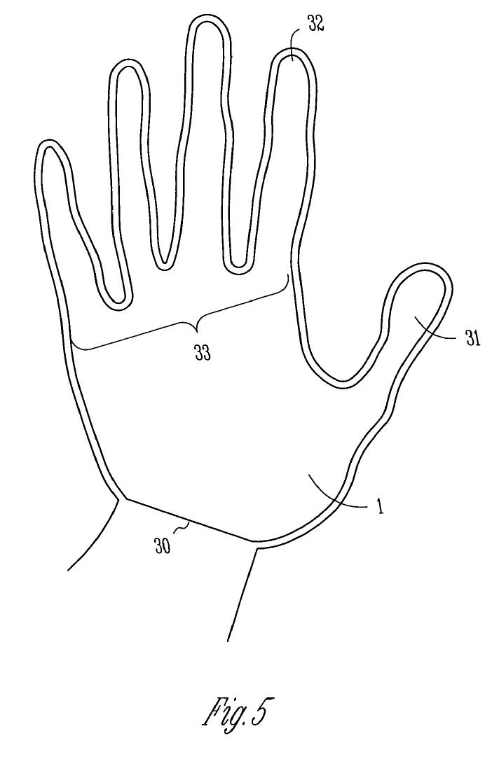

In another specific embodiment of the present invention, the adhesive skin patch 1 may be in the shape of the entire dorsal portion of a hand from the wrist 30 to the finger tips 32 and tip of the thumb 31, including the finger nails 34 (see FIG. 5)

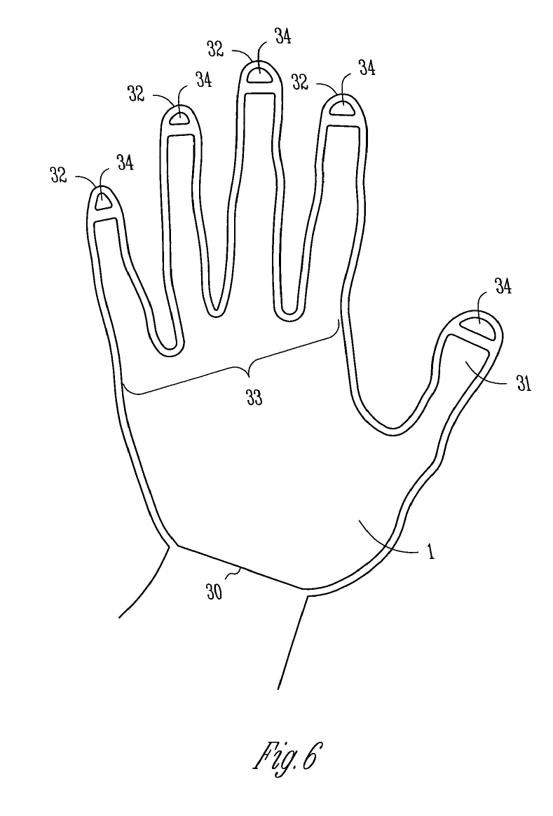

In another specific embodiment of the present invention, the adhesive skin patch 1 may be in the shape of the dorsal portion of a hand from the wrist 30 up to the finger nails 34, but not including the finger nails 34 (see FIG. 6)

In one embodiment, the adhesive skin patch 1 may be individually wrapped. Some consumers have shown a preference for adhesive skin patches that are individually wrapped. The individually wrapped adhesive skin patch 1 offers to the consumer the ability and convenience of being able to carry a few (e.g., 1, 2, or 3) adhesive skin patches 1 that are each individually wrapped. In such an embodiment, the use of one patch will not compromise the cleanliness and/or sterility of the remaining patches. Alternatively, more than one adhesive skin patch 1 may be wrapped together. For example, 2 to about 20, 2 to about 15, or 2 to about 10 adhesive skin patches 1 may be wrapped together. The cost of such packaging and wrapping may be decreased, compared to skin patches 1 that are individually wrapped. The cost of having two or more patches wrapped together is typically less expensive than skin patches 1 that are individually wrapped.

In one embodiment of the present invention, the adhesive patch 1 is sterile. The adhesive patch 1 may be sterilized by any suitable means known to those of skill in the art. For example, the adhesive patch 1 of the present invention may be sterilized by irradiation. Specifically, the adhesive patch 1 of the present invention may be sterilized by terminal irradiation (e.g., when the adhesive patch 1 of the present invention is in the package).

The adhesive patch 1 of the present invention may be formulated or manufactured employing the above components. The adhesive patch 1 of the present invention may be formulated or manufactured using any suitable technique. In one embodiment, the adhesive patch 1 may be formulated or manufactured as described in U.S. Pat. No. 5,536,263; U.S. Pat. No. 5,741,510; and references cited therein.

The adhesive patch 1 may be applied to any surface of a patient or to any surface of an article of clothing or a personal item worn by a patient. The adhesive patch 1 may be applied by the patient him/herself, or by another person (e.g., parent). The patient may use the adhesive patch 1 by rubbing his/her hands across the back side 4 of the backing 2 of the patch 1. Rubbing the back side 4 of the backing 2 of the patch 1 may cause a portion of the second formulation 24 to be deposited on the hand that is rubbing. The second formulation 24 may then be deposited on the non-rubbing hand or another skin surface through subsequent rubbing. In one embodiment, the patient may wear two adhesive patches 1, one on the dorsal aspect of each hand.

All publications, patents, and patent documents cited herein are incorporated by reference herein, as though individually incorporated by reference. The invention has been described with reference to various specific embodiments and techniques. However, it should be understood that many variations and modifications may be made while remaining within the spirit and scope of the invention.

1. A topical adhesive patch comprising:
a flexible backing having a front side and a back side;
a first formulation positioned on at least a portion on the front side of the backing, in at least a portion on the front side of the backing, or on and in at least a portion on the front side of the backing;
a second formulation positioned on at least a portion on the back side of the backing, in at least a portion on the back side of the backing, or on and in at least a portion on the back side of the backing;
the first formulation comprises an adhesive; and
the second formulation comprising an antibiotic agent, anti-fungal agent, anti-viral agent, or combination thereof.

2. The adhesive patch of claim 1 wherein the second formulation comprises an anti-viral agent selected from the group of zinc, lysine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, viracea2, cytovene, famciclovir, valaciclovir, penciclovir, nonoxynol-9, pharmaceutically acceptable salts thereof, and combinations thereof.

3. The adhesive patch of claim 1 wherein the second formulation comprises an anti-viral agent selected from the group of a hypochloride, a hypochloride generating compound, a peroxide, a peroxide generating compound, an organic halide, an organic halide generating compound, and combinations thereof.

4. The adhesive patch of claim 1 wherein the second formulation comprises an antibiotic agent selected from the group of cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole, sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, ascariasis, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, triclosan, pharmaceutically acceptable salts thereof, and combinations thereof.

5. The adhesive patch of claim 1 wherein the second formulation comprises an anti-fungal agent selected from the group of:
[1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-Amino-3,6-dideoxy-β-D-mannopyranosyl)oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid (Amphotericin B);
5-fluorocytosine (Flucytosine);
2,4-difluoro-α,α¹-bis(1H-1,2,4-triazol-1-ylmethyl)benzyl alcohol) (Fluconazole);
griseofulvin microsize (Griseofulvin);
(E)-N-(6,6-dimethyl-2-hepten-4-ynyl)-N-methyl-1-naphthalenemethanamine hydrochloride) (Terbinafine);
cis-1-acetyl-4-[4-[(2-(2,4-dichlorophenyl)-2-(1H-imadazol-1-ylmethyl)-1,3-dioxolan-4-yl]methoxyl]phenyl]piperazine (Ketoconazole);
(∀)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-ylimethyoxy]phenyl]-1-piperazinyl]phenyl]-Δ²-1,2,4-triazolin-5-one mixture with (∀)-1-[(R*)-sec-butyl]-4-[p-[4-[p-[[(2S*,4R*)-2-(2,4-dichlorophenyl)-2-(1H-1,2,4-triazol-1-ylmethyl)-1,3-dioxolan-4-ylimethoxy]phenyl]-1-piperazinyllphenyl]-Δ²-1,2,4-triazolin-5-one or (∀)-1-[(RS)-sec-butyl]-4-[p-[4-[p-[[(2R,4S)-2-(2,4-dichlorophenyl)-2-(1H-1,2,4-triazol-1-ylmethyl)-1,3-dioxolan-4-yl]-methoxy]phenyl]-1-piperazinyllphenyl]-Δ²-1,2,4-triazolin-5-one (Itraconazole);
2-chloro-5-hydroxy-1,3-dimethylbenzene (Chloroxylenol);
griseofulvin ultramicrosize (Griseofulvin);
(E)-N-(6,6,-dimethyl-2-hepten-4-ynyl)-N-methyl-1-naphthalenemanamine hydrochloride (Terbinafine);
6-cyclohexyl-1-hydroxy-4-methyl-2(1H)-pyridinone (Ciclopirox);
N-4-tert-butyl-benzyl-N-methyl-1-naphthalenemethylamine hydrochloride (Butenafine hydrochloride);
nystatin;
(E)-N-(Cinnamyl-N-methyl-1-naphthalenemethylamine hydrochloride (Naftifine hydrochloride);
1-(2,4-dichlorophenyl)-2-(1H-imidazol-1-yl)-O-[(2,4-dichlorophenyl)methyl]oxime, (Z)-, mononitrate (Oxiconazole nitrate),
6-cyclohexyl-1-hydroxy-4-methyl-2(1H)-pyridone (Ciclopirox);
selenium sulfide;
(∀)-1-[4-(p-chlorophenyl)-2-[(2,6-dichlorophenyl)thio]butyl]imidazole mononitrate (Butoconazole nitrate);
1-(o-Chloro-α,α-diphenylbenzyl)imidazole (Clotrimazole);
(cis-1-[p-[[2-(2,4-dichlorophenyl)-2-(1H-1,2,4-triazol-1-ylmethyl)-1,3-dioxolan-4-yl]methoxy phenyl]-4-isopropyl-piperazine (Termayazole);
6-cyclohexyl-1-hydroxy-4-methyl-2(1H)-pyridone (ciclopirox);
5-chloro-2-(2,4-dichlorophenoxy)phenol (triclosan);
and combinations thereof.

6. The adhesive patch of claim 1 wherein the flexible backing of the patch is porous.

7. The adhesive patch of claim 1 wherein the flexible backing is non-porous.

8. The adhesive patch of claim 1 wherein the flexible backing of the patch is vapor permeable.

9. The adhesive patch of claim 1 wherein the flexible backing of the patch comprises a non-woven fabric.

10. The adhesive patch of claim 1 wherein the flexible backing of the patch comprises polycellulose fibers, polyester fibers, polyurethane fibers, polyolefin fibers, polyamide fibers, cotton fibers, copolyester fibers, films, or any mixture thereof.

11. The adhesive patch of claim 1 wherein the backing of the patch comprises open cell foam.

12. The adhesive patch of claim 11, wherein the open cell foam comprises polyurethane, polyvinyl chloride, polyethylene, or any combination thereof.

13. The adhesive patch of claim 1 wherein upon contact with skin, the flexible backing of the patch retains the first formulation and the second formulation, and the adhesive patch allows moisture from the skin to pass through the adhesive patch.

14. The adhesive patch of claim 1 wherein the adhesive is an acrylic ester copolymer, a water-based adhesive, a hot melt adhesive, a pressure sensitive adhesive, a solvent based pressure sensitive adhesive, a polyacrylate, a polyisobutylene, a polybutene, a rubber, a silicone based pressure sensitive adhesive, a polystyrene-polybutadiene-polystyrene block polymer, a polystyrene-polyisoprene-polystyrene block polymer, a polystyrene-poly(ethylene-butylene)-polystyrene block polymer, or any combination thereof.

15. The adhesive patch of claim 14 wherein the adhesive is an acrylic ester copolymer.

16. A method of preventing or inhibiting the transmission of a disease selected from a bacterial disease, a fungal disease, a viral disease, and combinations thereof, in a mammal at risk of such disease, the method comprising topically contacting a skin surface of the mammal with a topical adhesive patch of claim 1, effective to prevent the transmission of the disease;
wherein the disease is selected from a bacterial disease, a fungal disease, a viral disease, and combinations thereof.

17. The method of claim 16 wherein the bacterial disease comprises bacterial meningitis, conjunctivitis, bacterial pneumonia, pertussis, tonsillitis, infectious diarrhea, cellulitis, impetigo, folliculitis, scalded skin syndrome, urinary tract infections, and combinations thereof.

18. The method of claim 16 wherein the viral disease comprises the common cold (rhinovirus), human flu (Influenzavirus A, Influenzavirus B, or Influenzavirus C), bronchiolitis, croup, measles, mumps, ruebella, influenza, infectious diarrhea, encephalitis, conjunctivitis, chicken pox, west nile virus, mononucleosis, cold sores, Avian influenza A (H5N1) virus, and combinations thereof.

19. The method of claim 16 wherein the fungal disease comprises ringworm, athlete's foot, yeast infections, and combinations thereof.

20. The method of claim 16 wherein the mammal is a human that is younger than 18 years of age.
